(19) United States
(12) Patent Application Publication
(10) Pub. No.: US 2025/0160747 A1
(43) Pub. Date: May 22, 2025

(54) SENSORS WITH ADHESIVE REGIONS

(71) Applicant: COVIDIEN LP, Mansfield, MA (US)

(72) Inventors: Derek L. Moody, Lafayette, CO (US); Alexander G. Breakstone, Boulder, CO (US); Winborne Hamlin, Westminster, CO (US); Abel Valdes, Lafayette, CO (US); Sarah L. Hayman, Boulder, CO (US)

(21) Appl. No.: 18/926,862

(22) Filed: Oct. 25, 2024

Related U.S. Application Data
(60) Provisional application No. 63/672,686, filed on Jul. 17, 2024.
(60) Provisional application No. 63/599,913, filed on Nov. 16, 2023.

(51) Int. Cl. A61B 5/00

(52) U.S. Cl. CPC A61B 5/68335; A61B 2560/045; A61B 2562/164; A61B 2562/185

(57) ABSTRACT

A sensor includes a light-emitting diode (LED), a detector to detect light emitted by the LED, and a body that supports the LED and the detector. The body includes a first bandage including a respective surface with a first type of adhesive and a second bandage coupled to a portion of the first bandage and including a respective surface with a second type of adhesive.

### CROSS-REFERENCE TO RELATED APPLICATIONS

This application claims priority to and the benefit of U.S. Provisional Application No. 63/672,686, entitled “SENSORS WITH ADHESIVE REGIONS,” filed on Jul. 17, 2024, and U.S. Provisional application No. 63/599,913, entitled “SENSORS WITH ADHESIVE REGIONS” and filed on Nov. 16, 2023, which are hereby incorporated by reference in their entireties for all purposes.

### TECHNICAL FIELD

The present disclosure generally relates to medical monitoring devices (e.g., sensors) that include adhesive regions to enable adherence to a surface and/or adherence for reapplication to the surface after removal.

### BACKGROUND

Various medical monitoring devices may be used to monitor physiological characteristics of an individual. For example, various sensors may be used to measure temperature, pressure, oxygen, and other physiological characteristics of the individual. One such sensor, a pulse oximetry sensor, may be used to measure oxygen saturation levels in blood of the individual by utilizing wavelengths of light. In this manner, the pulse oximetry sensor may provide physiological parameters related to respiratory and circulatory systems of the individual.

In certain cases, the pulse oximetry sensor may include an adhesive to enable application or adherence (e.g., attachment) to skin of the individual. After the application to the skin, the pulse oximetry sensor may emit light through the skin to measure the oxygen saturation levels in the blood of the individual.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it may be understood that these statements are to be read in this light, and not as admissions of prior art.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In certain embodiments, a sensor includes a light-emitting diode (LED), a detector to detect light emitted by the LED, and a body that supports the LED and the detector. The body includes a first bandage including a respective surface with a first type of adhesive and a second bandage coupled to a portion of the first bandage and including a respective surface with a second type of adhesive.

In certain embodiments, a sensor includes an LED, a detector to detect light emitted by the LED, and a body that supports the LED and the detector. The body includes a first bandage including a first zone of a first adhesive and a second bandage including a second zone of a second adhesive.

In certain embodiments, a system includes a sensor with a detector to generate sensor data indicative of a physiological parameter of a patient, wherein the sensor includes a body to support the detector. The body includes a first bandage with a first zone of a first adhesive, and the body includes a second bandage with a second zone of a second adhesive. The system also includes a monitor communicatively coupled to the sensor to receive and process the sensor data to determine the physiological parameter of the patient.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a perspective view of an embodiment of a medical monitoring system configured to monitor oxygen saturation, in accordance with an aspect of the present disclosure;

FIG. 2 is a block diagram of the medical monitoring system of FIG. 1, in accordance with an aspect of the present disclosure;

FIG. 3 is a perspective exploded view of an embodiment of a sensor, which may be employed in the medical monitoring system of FIG. 1, in accordance with an aspect of the present disclosure;

FIG. 4 is a bottom view of the sensor of FIG. 3 after assembly, in accordance with an aspect of the present disclosure;

FIG. 5 is a bottom view of an embodiment of a sensor, which may be employed in the medical monitoring system of FIG. 1, in accordance with an aspect of the present disclosure; and

FIG. 6 is an example illustration of a patient wearing a sensor that represents the sensor of FIG. 3 and the sensor of FIG. 5, in accordance with an aspect of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

It is presently recognized that it may be desirable to remove and reapply a sensor for various reasons, such as skin irritation, movement of a patient, inspection or maintenance of the sensor, and so forth. Without embodiments disclosed herein, the removal and reapplication of the sensor may result in discomfort for the patient and/or a loss of adhesive strength. Further, without embodiments disclosed herein, moisture (e.g., sweat) may accumulate at the sensor and may also cause the loss of adhesive strength. The loss of adhesive strength may reduce effectiveness and/or accuracy of the sensor.

Accordingly, the present disclosure generally relates to the field of medical monitoring devices, and more particularly, to a sensor. The sensor includes a body with multiple layers, such as a backing, a first light blocking layer (e.g., a metallized tape), and a second light blocking layer. The backing and the first light blocking layer may be coupled together (e.g., via any suitable adhesive) to form a first bandage (e.g., a top bandage). The second light blocking layer may form or be referred to herein as a second bandage (e.g., a bottom bandage). The first bandage and the second bandage may be coupled together (e.g., via any suitable adhesive) to form the body of the sensor. A flexible circuit that includes an emitter and a detector may be positioned between the first bandage and the second bandage as part of the sensor.

The body may include or provide multiple adhesive portions (e.g., zones, regions). In particular, a first adhesive (e.g., a first type of adhesive) may be applied to a respective first surface of the first bandage to provide a first adhesive portion, and a second adhesive (e.g., a silicone adhesive; a second type of adhesive, different than the first type of adhesive) may be applied to a respective first surface of the second bandage to provide a second adhesive portion. The first adhesive may facilitate repositioning by attaching the first bandage to itself via the first adhesive portion. For example, the first adhesive on the respective first surface of the first bandage may be separated and re-attached to a respective second surface of the first bandage to attach the first bandage to itself via the first adhesive portion at various times. The second adhesive may facilitate sensor adherence to a patient (e.g., skin and/or nail of the patient) where the second adhesive portion is in contact with the patient, and may be repositionable. For example, the second adhesive may contact and attach to the patient, while also allowing for separation and re-attachment to the patient at various times.

As described herein, the first bandage may include the backing and the first light blocking layer. The backing may include a porous material (e.g., polyethylene, polypropylene). The porous material may include through holes (e.g., extend through the respective first surface of the first bandage and through the respective second surface of the first bandage) through the porous material. The through holes may also extend through the first adhesive of the first adhesive portion. The porous material may facilitate self-attachment and re-attachment via the first adhesive portion, as well as breathability while applied to the patient. For example, the first bandage may wrap around a finger of the patient, and the backing may self-attach via the first adhesive portion. The porous material may limit or reduce (e.g., relative to non-porous material devoid of the through holes) a surface area of contact of the first adhesive while the backing is self-attached. That is, the porous material may provide sufficient retention and adhesive cohesion to self-attach at initial application(s), while also enabling separation and re-attachment with sufficient retention and adhesive cohesion due to the through holes that limit the surface area of contact of the first adhesive while the backing is self-attached at subsequent application(s).

The first light blocking layer may include the metallized tape, which may obstruct (e.g., block, suppress) light. Moreover, the first light blocking layer may cover or overlap a portion (e.g., a fraction) of the backing to limit or reduce (e.g., relative to certain existing sensors) a metallized footprint and improve breathability. The first light blocking layer may also include a tear portion that provides tear protection. The tear portion may include a first portion of metallized tape disposed along a first side of the first light blocking layer and a second portion of metallized tape disposed along a second side of the first light blocking layer. The tear portion may provide additional support to areas where the first bandage (e.g., the backing of the first bandage) may be susceptible to tear. As such, the sensor described herein may improve comfort, breathability, repositionability, and risk of tear by providing a combination of structural features, such as materials (including adhesives) at particular portions of the sensor, through holes, a limit in the metallized footprint, and the tear portion, for example.

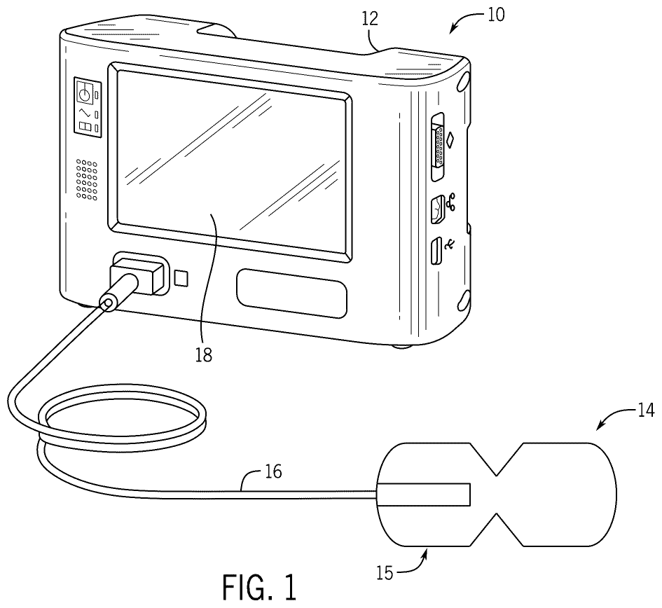

With the foregoing in mind, FIG. 1 is a perspective view of an embodiment of a medical monitoring system 10 that includes a patient monitor 12 (also referred to herein as a monitor for convenience) that may be used in conjunction with a medical sensor 14 (also referred to herein as a sensor for convenience). In the illustrated example, the monitor 12 is a pulse oximetry monitor and the sensor 14 is a pulse oximetry sensor. In such cases, the monitor 12 may be configured to process photoplethysmography (PPG) signals to calculate oxygen saturation (SpO2). It should be appreciated that the medical monitoring system 10 may be configured to obtain any of a variety of medical measurements and the techniques described herein may be adapted for use with any variety of monitors and sensors. By way of non-limiting example, in some embodiments, the monitor 12 may include a regional oximeter and the sensor 14 may include a regional saturation sensor. In such cases, the monitor 12 may be configured to process the PPG signals to calculate regional oxygen saturation (rSO₂). Additionally, although the depicted embodiments illustrate the sensor 14 configured for use on a patient's finger, it should be understood that the sensor 14 may be adapted for use at other tissue locations, such as a forehead, temple, earlobe, toe, foot, heel, ankle, stomach, chest, back, neck, write, thigh, or any other suitable measurement site (e.g., with pulsatile arterial flow).

The sensor 14 includes a sensor body 15 that includes multiple layers, such as a backing, a first light blocking layer (e.g., a metallized tape), and a second light blocking layer. The sensor body 15 may also include or support a flexible circuit with various components. The sensor 14 may be reusable, disposable, partially usable, or partially disposable. In certain embodiments, the medical monitoring system 10 may include multiple sensors 14 at multiple locations.

The sensor 14 is communicatively coupled to the monitor 12. In the illustrated embodiment, the sensor 14 is coupled to the monitor 12 via a cable 16. The cable 16 may interface directly with the sensor 14 and may include multiple conductors (e.g., wires) to transmit signals and/or receive signals. Additionally or alternatively, the sensor 14 may communicate with the monitor 12 wirelessly (e.g., the sensor 14 and the monitor 12 include wireless transceivers configured to communicate via any suitable wireless protocol). For example, the sensor 14 may include a transceiver that enables wireless signals to be transmitted to and/or received from an external device (e.g., the monitor 12). Additionally, the multiple conductors or the transceiver may transmit a raw digitized detector signal, a processed digitized detector signal, or a calculated physiological parameter, as well as any data that may be stored in the sensor 14. In operation, the monitor 12 may receive a signal from the sensor 14, and the monitor 12 may be configured to calculate or measure one or more physiological parameters based on the signal. In particular, the monitor 12 may include a processor configured to execute code (e.g., stored in a memory of the monitor 12 or received from another device) for filtering and processing the signal from the sensor 14 to calculate physiological parameters, such as oxygen saturation. The monitor 12 may additionally or alternatively calculate any variety of physiological parameters, such as arterial blood oxygen saturation, regional or tissue oxygen saturation, pulse rate, respiration rate, blood pressure, blood pressure characteristic measure, autoregulation status, brain activity, or any other suitable physiological parameter.

Additionally, as illustrated in FIG. 1, the monitor 12 includes a display 18 configured to display one or more calculated physiological parameters, such as oxygen saturation. The display 18 may also display other information, such as instructions to charge the sensor 14, alarm indications, settings, and so forth. In certain embodiments, the display 18 may be a touch screen display. The monitor 12 may include various input components, such as the touch screen display, knobs, switches, keys and keypads, buttons, and so forth, to provide for operation and configuration of the monitor 12. The monitor 12 may also include one or more indicator lights and one or more speakers. The monitor 12 may also include additional slot(s) or wireless interfaces (e.g., channels) to connect to additional devices, such as additional sensors to monitor additional physiological parameters of the patient and/or to monitor physiological parameters of other patients at one time.

Furthermore, one or more functions of the monitor 12 disclosed herein may also be implemented directly in the sensor 14, or by any other suitable device. For example, in some embodiments, the sensor 14 may include one or more processing components configured to calculate physiological parameters, such as oxygen saturation. The sensor 14 may have varying levels of processing power, and may output data in various stages to the monitor 12. For example, in some embodiments, the data output to the monitor 12 may be analog signals, such as detected light signals (e.g., pulse oximetry signals or regional saturation signals), or processed data.

Further, in some embodiments, the sensor 14 may include a battery to provide power to components of the sensor 14. For example, the sensor 14 may be configured to operate in a wireless mode and, at times, may not receive power from the monitor 12 while operating in the wireless mode. In some embodiments, the battery may be a rechargeable battery such as, for example, a lithium ion, a lithium polymer, a nickel-metal hydride, a nickel-cadmium battery, or any other suitable rechargeable battery. In other embodiments, any suitable power source may be utilized, such as, one or more capacitors or an energy harvesting power supply (e.g., a motion generated energy harvesting device, thermoelectric generated energy harvesting device, or any other suitable energy harvesting power supply). As described herein, the sensor 14 includes various structural features to provide comfort, breathability, and repositionability, as well as to block tears (e.g., structural damage) to the sensor 14.

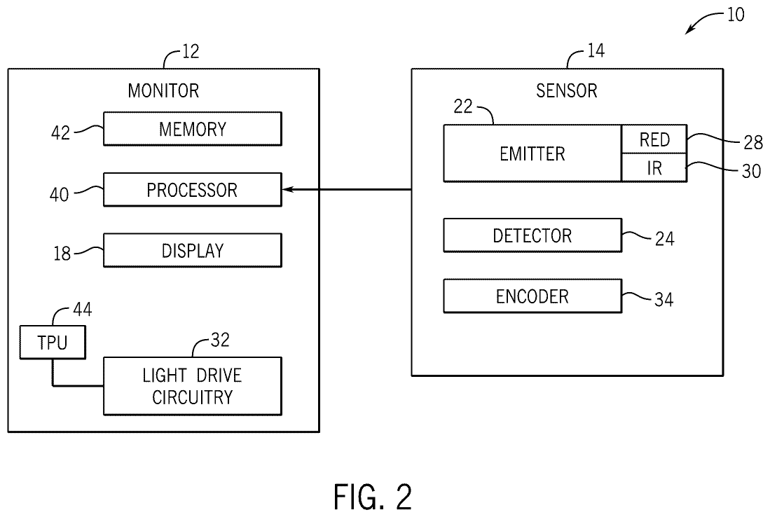

Turning to FIG. 2, a simplified block diagram of the medical monitoring system 10 is illustrated in accordance with an embodiment. The sensor 14 includes optical components in the form of one or more emitters 22 (referred to herein as an emitter for convenience) and one or more detectors 24 (referred to herein as a detector for convenience). The emitter 22 includes at least two light emitting diodes (LEDs) that are configured to emit at least two wavelengths of light, e.g., a red LED 28 configured to emit wavelengths of light within the red spectrum and an infrared (IR) LED 30 configured to emit wavelengths of light within the infrared or near infrared spectrum. In one embodiment, the LEDs 28, 30 emit light in a range of about 600 nanometers (nm) to about 1000 nm. In one embodiment, the red LED 28 is configured to emit light between approximately 600 nm and 735 nm, and the IR LED 30 is configured to emit light between approximately 800 nm and 1000 nm. It should be noted that the emitter 22 may also transmit 3, 4, or 5 or more wavelengths of light in any suitable application. For example, in a pulse oximetry application, the sensor 14 may include one emitter 22 with the red LED 28 and the IR LED 30 that are configured to emit two wavelengths of light. As another example, in a regional oximetry application, the sensor 14 may include two emitters 22 that each include a respective red LED 28 and a respective IR LED 30, and together the two emitters 22 are configured to emit two to four wavelengths of light

As discussed in more detail herein, a light drive circuitry 32 of the monitor 12 may provide respective drive currents to the LEDs 28, 30 to cause the LEDs 28, 30 to emit respective wavelengths of light. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

The emitter 22 emits light that passes through blood perfused tissue, and the detector 24 detects the light as reflected or transmitted by the tissue. The emitter 22 and the detector 24 may be arranged in a transmission configuration or a reflectance configuration with respect to one another. In the transmission configuration, the light enters the detector 24 after passing through the tissue of the patient. In the reflectance configuration, the light is reflected by elements in the tissue of the patient to enter the detector 24. In any case, the detector 24 may generate a signal (e.g., PPG signal) indicative of an intensity of the light received at the detector 24, and the detector 24 may send the signal to the monitor 12.

A signal representing light intensity versus time or a mathematical manipulation of this signal (e.g., a scaled version thereof, a log taken thereof, a scaled version of a log taken thereof) may be referred to as the PPG signal. Additionally, the term “PPG signal,” as used herein, may also refer to an absorption signal (e.g., representing an amount of light absorbed by the tissue) or any suitable mathematical manipulation thereof. The amount of light detected or absorbed may then be used to calculate any of a number of physiological parameters, including oxygen saturation (e.g., the saturation of oxygen in pulsatile blood, SpO2), an amount of a blood constituent (e.g., oxyhemoglobin), and/or a physiological rate (e.g., pulse rate or respiration rate; when each individual pulse or breath occurs). For SpO2, red and IR wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood, such as from empirical data that may be indexed by values of a ratio, a lookup table, from curve fitting, or other interpolative techniques.

As shown, the sensor 14 also includes an encoder 34. The encoder 34 may store information about the sensor 14, such as a type of sensor, calibration information, and so forth. When accessed by the monitor 12, the information about the sensor 14 may enable the monitor 12 to calculate oxygen saturation and/or other physiological parameters using the signal received from the detector 24. In certain embodiments, the sensor 14 may include sensing components in addition to, or instead of, the emitter 22 and the detector 24. For example, in one embodiment, the sensor 14 may include one or more actively powered electrodes (e.g., four electrodes) to obtain an electroencephalography signal.

As shown, the monitor 12 includes one or more processors 40, a memory 42, and the display 18. The processor 40 may process the signal received from the detector 24, such as by performing synchronized demodulation, amplification, and filtering of the signal. The processor 40 may process the signal received from the detector 24 to calculate one or more physiological parameters, such as the oxygen saturation, using various algorithms. Coefficients utilized in the algorithms may be accessed by the processor 40 from the encoder 34 or determined by the processor 40 based on the calibration information of the sensor 14, for example.

As shown, the monitor 12 includes a time processing unit (TPU) 44, which may be controlled by the processor 40 and is configured to provide timing control signals to the light drive circuitry 32 and optionally to other parts of the medical monitoring system 10. The light drive circuitry 32 may control when the red LED 28 and the IR LED 30 are illuminated and/or a drive current provided to the red LED 28 and the IR LED 30. It should be appreciated that one or more functions or components of the monitor 12 disclosed herein may also be implemented directly in the sensor 14, or by any other suitable device. As described herein, the sensor 14 includes various structural features, such as multiple adhesive portions, to provide comfort, breathability, and repositionability, as well as to block tears (e.g., structural damage) to the sensor 14.

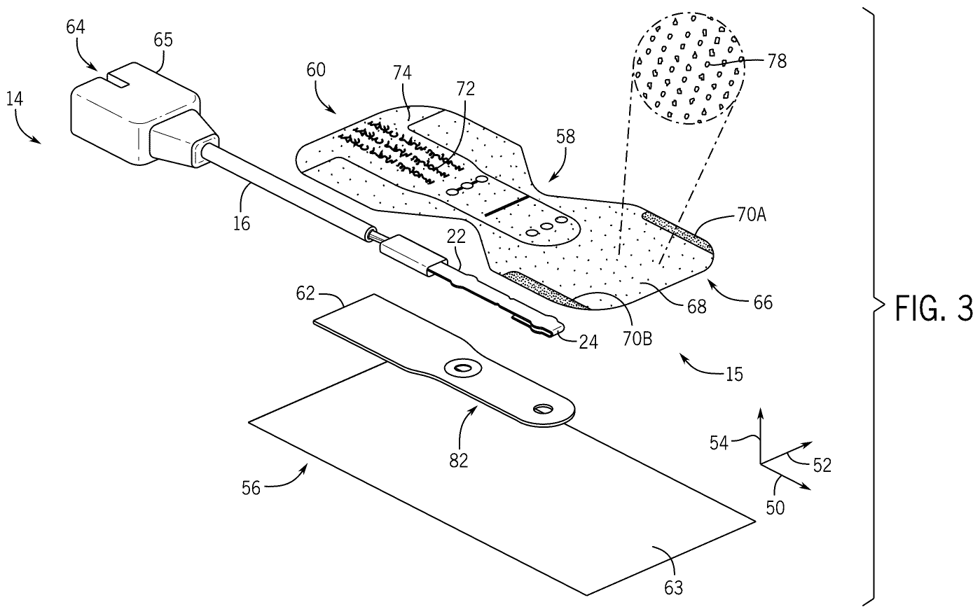

FIG. 3 is a perspective exploded view of an embodiment of the sensor 14, which may be employed in the medical monitoring system 10 of FIG. 1, in accordance with an aspect of the present disclosure. To facilitate discussion, the sensor 14 is described with reference to a longitudinal axis or direction 50, a lateral axis or direction 52, and/or a vertical axis or direction 54. Further, the sensor 14 is described with reference to a first side 56 (e.g., bottom side; patient-facing side) and a second side 58 (e.g., top side; opposite the first side 56, such as opposite the first side 56 along the vertical axis 54 at least when in a flat configuration, such as prior to application to a patient).

The sensor 14 includes the sensor body 15, which includes a first bandage 60 (e.g., a top bandage) that couples to a second bandage 62 (e.g., a bottom bandage). The first bandage 60 and the second bandage 62 are coupled together via any suitable adhesive. The sensor 14 is supported on a release liner 63 prior to application to the patient (e.g., at manufacturing; for storage prior to application to the patient).

The sensor body 15 houses or carries components of the sensor 14. For example, the sensor body 15 is provided around the emitter 22 and/or the detector 24. A flexible circuit 64 positioned between the first bandage 60 and the second bandage 62 includes the emitter 22 and the detector 24 and may be coupled to the cable 16, which may connect various components (including the emitter 22 and the detector 24) of the sensor 14 to a connector 65 (e.g., plug).

The first bandage 60 includes a first adhesive 66 at a respective first surface (e.g., a bottom surface) of the first bandage 60 to provide a first adhesive portion (e.g., a first zone or region; an outer zone). In certain embodiments, the first adhesive 66 may include an acrylic-based adhesive (e.g., acrylic adhesive; adhesive that includes acrylic polymers). The first adhesive 66 facilitates application and re-positioning on the patient by attaching the first bandage 60 to itself via the first adhesive portion. For example, the first bandage 60 may wrap around a finger of the patient and attach to itself via the first adhesive portion. The first bandage 60 may adapt (e.g., conform) closely to the finger of the patient upon self-adherence. Indeed, the first bandage 60 may adhere to itself upon contact between a respective second surface (e.g., top surface) and the first adhesive portion. In this manner, the first bandage 60 may securely stick to itself without additional fasteners (e.g., without separate tape, clips, or wraps).

The first bandage 60 includes a backing 68, a first light blocking layer 72 (e.g., a metallized tape) coupled to the backing 68, and a tear prevention portion 74 (e.g., tear reinforcement portion) extending from or included as part of the first light blocking layer 72. The backing 68 extends along the longitudinal axis 50 and the lateral axis 52 (e.g., at least in the flat configuration) and includes porous material (e.g., polyethylene, polypropylene). The porous material is a flexible material that includes through holes 78 (e.g., pores, voids, open spaces), which may enable a passage of liquid (e.g., sweat) and gases (e.g., air) through the porous material. The through holes 78 traverse the first bandage 60 along the vertical axis 54 (e.g., at least in the flat configuration). That is, the through holes 78 are open to and extend between the respective first surface of the first bandage 60 and the respective second surface of the first bandage 60. Thus, a continuous path (e.g., fluid path) is created between the respective first surface of the first bandage 60 and the respective second surface of the first bandage 60. It should be noted that the through holes 78 may have an irregular spacing, as shown in FIG. 3, or the through holes 78 may be arranged with uniform spacing (e.g., in rows and columns). In some embodiments, the porous material may include embossing to create a raised (e.g., raised and/or recessed; relief; textured) pattern at the respective first surface of the first bandage 60 and/or the respective second surface of the first bandage 60.

Further, the through holes 78 and/or the embossing may limit or reduce contact of the first adhesive 66 during self-adherence. Indeed, when the through holes 78 and/or the embossing come together at the self-adherence, the through holes 78 and/or the embossing may result in incomplete contact at the first adhesive 66, thereby facilitating subsequent separation for re-attachment and preserving adhesiveness of the first adhesive 66 for the re-attachment. Thus, the through holes 78 and/or the embossing enable spaces (e.g., air gaps) to persist between points of contact along the first adhesive 66 at the self-adherence, which may enable breathability and improve retention of adhesive cohesion upon positioning and re-positioning of the sensor 14. Additionally, when the sensor 14 is placed on the finger of the patient, the patient may exude sweat from sweat glands on the finger. The sweat may evaporate more rapidly through the through holes 78 formed in the porous material of the backing 68. In this way, at least the first adhesive portion of the first bandage 60 (e.g., defined by or including the first adhesive 66 on an exposed portion of the respective first surface of the backing 68 that does not overlap with or is not covered by the first light blocking layer 72) facilitates self-adherence and re-positioning of the sensor 14, and also provides breathability (e.g., sweat evaporation, air flow) via the porous material of the backing 68.

In some embodiments, the backing 68, the first light blocking layer 72, and/or the tear prevention portion 74 includes markers 70 (e.g., a first marker 70A and a second marker 70B). The markers 70 each provide an identifier (e.g., a visible reference point), which enables identification or recognition of one or more edges of the backing 68. As illustrated, the backing 68 includes the markers 70 (e.g., the first marker 70A and the second marker 70B) each disposed on opposite lateral sides (e.g., a left side, a right side) of the backing 68. The markers 70 may be distinct in color (e.g., a contrasting color relative to a remainder or at least adjacent portions of the backing 68), shape, length, width, and/or location to enable the identification or recognition. Thus, the identification or recognition of the one or more edges of the backing 68 enables (e.g., by an operator, such as the patient or a medical professional) an initiation of a process of pulling the backing 68 apart after the self-adherence. It should be noted that although FIG. 3 illustrates two markers, any suitable number of markers may be included in the backing 68 to enable the identification or recognition of the edges of the backing 68.

The first light blocking layer 72 is disposed underneath (e.g., below) the backing 68 and may be coupled to the respective first surface of the backing 68 via any suitable adhesive. The first light blocking layer 72 extends along the longitudinal axis 50 and the lateral axis 52 and obstructs (e.g., blocks, suppresses) light (e.g., blocks ambient light from reaching the detector 24). With reference to FIG. 3, a covered portion of the backing 68 that is positioned over the first light blocking layer 72 may include openings (e.g., the through holes 78 covered by the first light blocking layer 72; as a result of manufacturing techniques for the backing 68); however, it should be appreciated that at least some of the covered portion of the backing may be devoid of the openings. As the first light blocking layer 72 blocks light, the first light blocking layer 72 includes less breathable material relative to the backing 68. Thus, a reduction or limit in a size of the first light blocking layer 72 is implemented to increase or improve breathability for the patient. For example, the first light blocking layer 72 is a portion (e.g., a fraction) of a size of the backing 68 (e.g., extend across less than 80, 70, 60, or 50 percent of the backing 68 in the longitudinal direction 50 and the lateral direction 52; cover less than 80, 70, 60, 50, or 40 percent of a surface area of the backing 68). In this manner, the reduction or limit of the size of the first light blocking layer 72 increases or improves breathability for an area on the patient where the sensor 14 is placed.

The first light blocking layer 72 includes the tear prevention portion 74 extending along the lateral axis 52. The tear prevention portion 74 may be disposed in any suitable area of the first bandage 60. In some embodiments, the first bandage 60 may include a third adhesive at the respective first surface of the first bandage 60 corresponding to the tear prevention portion 74. For example, the third adhesive may include a rubber-based adhesive (e.g., adhesive that includes rubber). Moreover, the tear prevention portion 74 may include a first portion of metallized tape and a second portion of metallized tape. In some embodiments, the first portion of the metallized tape may be placed along a first side (e.g., a left side along the lateral axis 52) of the first light blocking layer 72 and the second portion of the metallized tape may be placed along a second side (e.g., a right side along the lateral axis 52) of the first light blocking layer 72. Additionally or alternatively, the tear prevention portion 74 may be disposed at or near the markers 70 and may include any suitable material that provides reinforcement for the first bandage 60. The tear prevention portion 74 provides reinforcement (e.g., support, strength, security) to the first bandage 60. Accordingly, the tear prevention portion 74 provides protection from damage, wear and tear, and external forces. In some embodiments, the tear prevention portion 74 is disposed where the cable 16 extends from or contacts the first bandage 60 to couple the sensor 14 to the monitor 12. In this manner, the tear prevention portion 74 reduces or limits a risk of tear during coupling and uncoupling of the sensor 14 to the monitor 12, and provides tear protection for the backing 68 during separation or re-positioning of the sensor 14 on the patient.

Additionally, as described herein, a second light blocking layer (e.g., a metallized tape) forms the second bandage 62, which is disposed beneath the first bandage 60. The first bandage 60 and the second bandage 62 are coupled together by coupling the first light blocking layer 72 to the second bandage 62 to one another via any suitable adhesive, and with the flexible circuit 64 positioned between the first bandage 60 and the second bandage 62. The second bandage 62 is the same size and shape (or similar in size and shape) as the first light blocking layer 72, and aligns or overlaps with the first light blocking layer 72. In this manner, the second light blocking layer also provides a reduced metallized footprint and improves breathability of the sensor 14. The second light blocking layer that forms the second bandage 62 obstructs or blocks light.

Further, a second adhesive 82 is applied to a respective first surface (e.g., a bottom surface, a surface closest to the patient, a patient-side surface, a patient-contacting surface, an exposed surface) of the second bandage 62 to provide a second adhesive portion (e.g., a second zone or region; an inner zone). The second adhesive 82 includes a silicone-based adhesive, such as a silicone gel or a silicone pressure sensitive adhesive. The second adhesive 82 facilitates adherence to the patient (e.g., to skin and/or nail of the patient) where the second adhesive portion is in contact with the patient. The second adhesive 82 may be any suitable thickness that enables adhesion to the patient. For example, the thickness may be any value between 0.1 millimeter (mm) and 1.5 mm. The second adhesive 82 may minimize disruption of the skin (e.g., by removing only minimal skin protein) during removal. Further, the silicone of the second adhesive 82 may provide comfort to the patient.

The first adhesive 66 (e.g., on the respective first surface of the backing 68; at the first adhesive portion) and the second adhesive 82 (e.g., on the respective first surface of the second bandage 62; at the second adhesive portion) may be different from one another (e.g., different types of adhesives, such as with different chemical formulations and/or different material properties). The first adhesive 66 may be selected to facilitate adhesion of the backing 68 to itself (as well as separation and re-attachment to itself), while the second adhesive 82 may be selected to facilitate adhesion of the second bandage 62 to the patient (as well as separation and re-attachment to the patient). Accordingly, the sensor 14 may provide multiple adhesive portions (e.g., zones, regions) to provide comfort, breathability, repositioning, and so forth.

The first adhesive 66 and the second adhesive 82 may also retain peel force (e.g., peel strength, peel adhesion), even after various removals and reapplication of the sensor 14. The peel force may be a measure of force involved in peeling apart the first adhesive 66 or the second adhesive 82 from the backing 68 or the patient, respectively. As such, the first adhesive 66 and the second adhesive 82 may be removed and reapplied (e.g., repositioned) multiple times while maintaining the peel force. For example, repositioning the sensor 14 may include separating the first adhesive 66 from itself, unwrapping the first bandage 60 from a portion of the patient, separating the second bandage 62 from the portion of the patient, and reapplying the sensor 14 to the patient.

It should be appreciated that the first light blocking layer 72 is coupled to the backing 68 via any suitable adhesive, such as the first adhesive 66. For example, during manufacturing, the first adhesive 66 may be applied across an entirety of the respective first surface of the backing 68, and then the first light blocking layer 72 may be coupled to the backing 68 via the first adhesive 66. Similarly, it should be appreciated that the first light blocking layer 72 is coupled to the second bandage 62 via any suitable adhesive, such as the first adhesive 66. For example, during manufacturing, the first adhesive 66 may be applied across an entirety of the respective first surface of the first light blocking layer 72 or the second bandage 62, and then the first light blocking layer 72 may be coupled to the second bandage 62 via the first adhesive 66.

As noted herein, prior to use by the patient, the first adhesive 66 and the second adhesive 82 are coupled to the release liner 63. The release liner 63 includes a material (e.g., paper, plastic) that enables coverage of the first adhesive 66 and the second adhesive 82 to prevent the first adhesive 66 and the second adhesive 82 from adhering (e.g., sticking) to other surfaces. For example, the release liner 63 may include a backing paper that provides protection for the first adhesive 66 and the second adhesive 82 until the release liner 63 is peeled off (e.g., removed) to expose the first adhesive 66 and the second adhesive 82 for use. After the release liner 63 is peeled off, the sensor 14 may be applied to the patient.

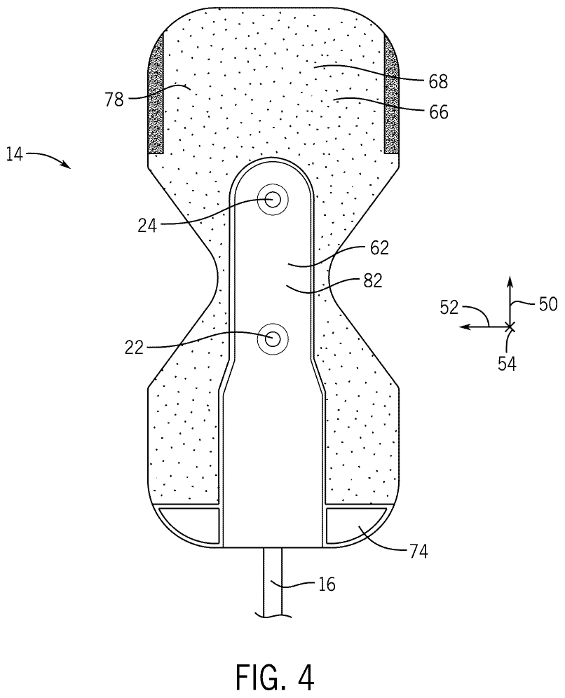

With the foregoing in mind, FIG. 4 is a bottom view of the sensor 14 after assembly, in accordance with an aspect of the present disclosure. To facilitate discussion, the sensor 14 is described with reference to the longitudinal axis or direction 50, the lateral axis or direction 52, and/or the vertical axis or direction 54. When the sensor 14 is assembled and in the flat configuration, the first adhesive region and the second adhesive region are exposed along respective first surfaces of the sensor 14. For example, when the release liner 63 of FIG. 3 is adhered to the sensor 14 in the flat configuration, the first adhesive region with the first adhesive 66 and the second adhesive region with the second adhesive 82 contact and adhere to the release liner 63. Then, when the release liner 63 is removed from the sensor 14, the first adhesive region with the first adhesive 66 and the second adhesive region with the second adhesive 82 are subject to environmental exposure at least when the sensor 14 is in the flat configuration and before application to the patient. In particular, the first adhesive region includes the first adhesive 66 exposed on the exposed portion of the respective first surface of the backing 68 of the first bandage 60 (e.g., that does not overlap with or is not covered by the second bandage 62; the second bandage 62 only covers a portion of the first bandage 60), and the second adhesive region includes the second adhesive 82 exposed along the respective first surface of the second bandage 62. In this way and as shown, the first adhesive region with the first adhesive 66 at least partially surrounds and extends outwardly from (e.g., along the longitudinal axis 50 and the lateral axis 52) the second adhesive region with the second adhesive 82, which may effectively provide or form the outer adhesive zone and the inner adhesive zone. As shown, the second bandage 62 includes openings aligned with the emitter 22 and the detector 24.

As described herein, the first adhesive portion (e.g., the backing 68) includes the porous material, which includes the through holes 78. The through holes 78 may reduce a surface area of contact of the first adhesive 66 when the backing 68 adheres itself by enabling the spaces (e.g., holes) to persist around the points of contact. To apply the sensor 14 to the patient, the second bandage 62 may be adhered to a portion of a finger of the patient (including a nail of the finger of the patient) via the second adhesive 82 at the second adhesive portion. Then, the first bandage 60 may be wrapped around the finger of the patient and attach to itself via the first adhesive 66 at the first adhesive portion. For example, the sensor 14 may fold over a tip of the finger of the patient, and a first flap (e.g., on a left side) of the first bandage 60 and a second flap (e.g., on a right side) of the first bandage 60 may each wrap around sides of the finger and self-adhere via the first adhesive 66 at the first adhesive portion (e.g., the respective second surface of the first bandage 60 may adhere to the first adhesive 66 at the first adhesive portion on the respective first surface of the first bandage 60). The through holes 78 may limit or reduce the surface area of the contact of the first adhesive portion, improving adhesive retention when re-positioning or reapplying the sensor 14.

Further, it should be appreciated that the second bandage 62 (e.g., the second light blocking layer) is coupled to the first light blocking layer to support the flexible circuit 64. As shown, the second bandage 62 is the same (e.g., or similar) in size and shape as the first light blocking layer.

As illustrated in FIG. 4, the tear prevention portion 74 includes metallized tape disposed on opposite sides (e.g., a right side and a left side) of the first light blocking layer and the second bandage 62. The tear prevention portion 74 is disposed where the cable 16 extends from the sensor 14. It should be noted that although the tear prevention portion 74 is described as being disposed where the cable 16 couples to the sensor 14, the tear prevention portion 74 may be disposed in any suitable area with a high probability of tear of the first bandage 60 or the second bandage 62. For example, the tear prevention portion 74 may be disposed at or near the markers 70. Further, it should be noted that although the tear prevention portion 74 is described as including the metallized tape, the tear prevention portion 74 may include any suitable material that provides reinforcement for the first bandage 60 or the second bandage 62.

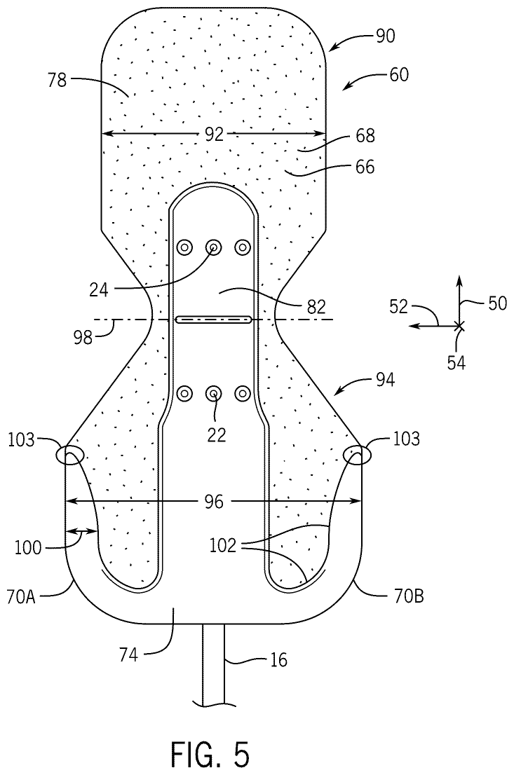

FIG. 5 is a bottom view of an embodiment of the sensor 14, which may be employed in the medical monitoring system 10 of FIG. 1, in accordance with an aspect of the present disclosure. To facilitate discussion, the sensor 14 is described with reference to the longitudinal axis or direction 50, the lateral axis or direction 52, and/or the vertical axis or direction 54. The sensor 14 may include a first end portion 90 (e.g., distal end portion furthest from the cable 16) extending along the longitudinal axis 50 and the lateral axis 52 with a first width 92 (e.g., a maximum width) extending along the lateral axis 52. Moreover, the sensor 14 may include a second end portion 94 (e.g., proximal end portion closest to the cable 16) extending along the longitudinal axis 50 and the lateral axis 52 with a second width 96 (e.g., a maximum width) extending along the lateral axis 52.

The first width 92 may be different (e.g., smaller or larger) than the second width 96. For example, as illustrated in FIG. 5, the first width 92 is smaller than the second width 96. Therefore, the sensor 14 may have an asymmetrical configuration (e.g., shape, arrangement; about a midline 98 that extends in the lateral direction 52). During application of the sensor 14 to the patient, the asymmetrical configuration may provide guidance to the patient and/or the medical professional as to how to apply the sensor 14 to the patient. That is, the patient and/or the medical professional may be led or guided by the asymmetrical configuration to begin application of the sensor 14 by applying first end portion 90 including the first width 92 based on the first width 92 being smaller than the second width 96. That is, the first end portion 90 of the sensor 14 may be wrapped around and/or adhered to at least a portion of the finger of the patient via the first adhesive 66 and/or the second adhesive 82 at the first end portion 90 of the sensor 14.

The patient and/or the medical professional may subsequently apply the second end portion 94 including the second width 96 based on the second width 96 being larger than the first width 92. Indeed, a first flap (e.g., on a left lateral side) of the second end portion 94 and a second flap (e.g., on a right lateral side) of the second end portion 94 may each wrap around sides of the finger and over the first end portion 90 (e.g., over the respective second surface of the first bandage 60). Thus, the second end portion 94 of the sensor 14 may be wrapped around and/or adhered to at least a portion of the finger of the patient via the first adhesive 66 and/or the second adhesive 82 at the second end portion 94 of the sensor 14. Further, the second end portion 94 of the sensor 14 may also be wrapped around and/or adhered to at least a portion of the first end portion 90 via the first adhesive 66 (e.g., the respective first surface, or bottom surface, of the second end portion 94 may contact and adhere to the respective second surface, or top surface, of the first end portion 90 via the first adhesive 66 on the respective first surface of the second end portion 94). After application of the sensor 14 to the patient, the first end portion 90 may be on a first side (e.g., generally centered on a bottom side, at a palm side of the hand) of the finger of the patient and the second end portion 94 may be on a second side (e.g., generally centered on a top side, at a dorsal side of the hand) of the finger of the patient.

Advantageously, in addition to guiding application of the sensor 14, the first end portion 90 including the first width 92 (e.g., smaller width) may block contact between the first adhesive 66 at the respective first surface of the first end portion 90 and the first adhesive 66 at the respective first surface of the second end portion 94. Accordingly, such features of the sensor 14 may provide improved adhesion to the patient, a comfortable fit on the patient, and/or facilitate repositioning of the sensor 14, for example.

As illustrated in FIG. 5, the tear prevention portion 74 may include the metallized tape disposed along (e.g., bordering) a first edge (e.g., first edge portion; closest to the cable 16) of the second end portion 92. In some embodiments, the tear prevention portion 74 may include a width 100 (e.g., along the lateral axis 52) of at least 2, 3, 4, 5, or more millimeters (mm), such that the tear prevention portion 74 provides sufficient support to block tearing of the first bandage 60, while also allowing the first bandage to provide breathability via the porous material, as described herein. As shown, the tear prevention portion 74 may include curved edges 102 to facilitate manufacturing and/or to reduce peeling (e.g., separation) of the tear prevention portion 74 from the backing 68. It should be noted that any suitable edge of the tear prevention portion 74 may include any suitable shape. For example, at least part of the curved edges 102 (e.g., an end or tip portion, shown within region 103 in FIG. 5; proximate to the emitter 22 along the longitudinal axis 50) may include a square shape (e.g., straight, non-curved shape) or other curved shape (e.g., with curvature other than the curved edges 102 shown in FIG. 5) to increase or improve adhesion and/or to reduce peeling (e.g., separation) of the tear prevention portion 74 from the backing 68, particularly during removal of the sensor 14 from the release liner 63 or the patient.

Further, the first marker 70A and the second marker 70B may be disposed on (e.g., overlapping; on a top side) or close to the tear prevention portion 74, such as disposed on opposite lateral regions of the tear prevention portion 74 on opposite lateral sides of the second end portion 94 of the sensor 14. For example, the first marker 70A may be disposed on the first flap of the second end portion 94 and the second marker 70B may be disposed on the second flap of the second end portion 94. In the bottom view of the sensor 14 in FIG. 5, the first marker 70A and the second marker 70B are not visible as they are positioned on the tear prevention portion 74, but their locations are indicated via respective line indicators to respective edges of the first marker 70A and the second marker 70B.

The first marker 70A may enable identification of the first flap of the second end portion 94 and the second marker 70B may enable identification of the second flap of the second end portion 94. As such, the markers 70 may enable an initiation of a process of pulling back the first flap of the second end portion 94 and the second flap of the second end portion 94 for removal of the sensor 14. Moreover, placement of the markers 70 on or close to the tear prevention portion 74 may reduce or limit a risk of tear during removal of the sensor 14 (e.g., block tearing during removal of the sensor 14). In addition, it should be noted that the first adhesive region with the first adhesive 66 may include the porous material, which includes the through holes 78 to enable breathability of the sensor 14. Indeed, it should be appreciated that the sensor 14 shown in FIG. 5 may include any features disclosed herein, such as the emitter 22 and the detector 24, for example.

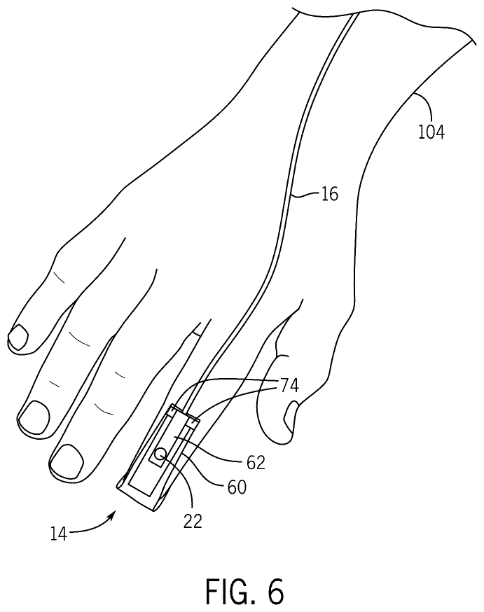

FIG. 6 is an example illustration of a patient 104 wearing the sensor 14, in accordance with an aspect of the present disclosure. In some embodiments, the sensor 14 is oriented such that a mid-point of the sensor 14 is aligned with a tip of a finger on a hand of the patient 104. The sensor 14 folds over the finger to position a first portion (e.g., with the cable 16) along a top of the finger and to position a second portion along a bottom of the finger (e.g., a palm side of a hand). The sensor 14 includes the tear prevention portion 74 disposed on each side of the cable 16 to provide the tear protection.

In a transmission configuration, the detector 24 is placed opposite the emitter 22. The emitter 22 emit light, and the detector 24 detects the light after transmission through the finger of the patient 104. The detector 24 generates the signal indicative of oxygen saturation (SpO₂) of the patient 104. As illustrated in FIG. 6, the first bandage 60 wraps around the finger of the patient 104 and adheres to itself, such as along the bottom of the finger of the patient 104 (e.g., at the palm side of the hand). The second bandage 62 adheres directly to the skin and/or nail of the patient 104.

As described herein, in some embodiments, the sensor 14 may include the first end portion 90 including the first width 92 and the second end portion 94 including the second width 96. The sensor 14 folds over the finger to wrap the first end portion 90 around the finger. Moreover, the first flap of the second end portion 94 and the second flap of the second end portion 94 wrap around the sides of the finger and over (e.g., at least partially surrounding) the first end portion 90. Thus, the first end portion 90 and the second end portion 94 may be positioned (e.g., arranged) on opposite sides of the finger.

Accordingly, embodiments described herein enable breathability of the sensor 14 by incorporating the backing 68 with porous material, which enables the passage of the gases and liquids. Further, the backing 68 with the porous material enables adjustability and improves repositionability by reducing direct contact of the first adhesive portion during self-adherence of the backing 68, thereby retaining adhesive cohesion and strength. Embodiments described herein also improve breathability by providing limited size for the first light blocking layer 72, which reduces the metallic footprint. Further, the tear portions 74 in areas with a relatively high probability of tear provides additional tear protection for the sensor 14, which may be particularly helpful in view of the porous material utilized for the backing 68 and the limited size of the first light blocking layer 72 (e.g., which may increase the probability of tear, if provided without the tear portions 74). As such, the breathability, repositionability, and provided tear protection for the sensor 14 may improve comfort and enable accurate measurements even after repositioning of the sensor 14. Accordingly, because of structural features disclosed herein, including multiple adhesive portions defined by any combination of the backing 68, the first light blocking layer 72, the tear portions 74, the second light blocking layer of the second bandage 62, and the adhesives, the comfort and effectiveness of the sensor 14 may be improved. It should be appreciated that features shown and described herein may be combined in any suitable manner, features shown and described herein may be omitted, and other features may be added.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

The invention may be further described by reference to the following examples:

Example 1: A sensor, including: a light-emitting diode (LED); a detector to detect light emitted by the LED; and a body that supports the LED and the detector, wherein the body includes: a first bandage including a respective surface with a first type of adhesive; and a second bandage coupled to a portion of the first bandage and including a respective surface with a second type of adhesive.

Example 2: The sensor according to example 1, wherein the first bandage includes a backing, and wherein the backing includes: the respective surface with the first type of adhesive; and a porous material with through holes that extend that the first type of adhesive.

Example 3: The sensor according to example 2, wherein the porous material includes polyethylene, polypropylene, or any combination thereof.

Example 4: The sensor according to any of examples 2 or 3, wherein the first bandage includes a first light blocking layer coupled to the backing, the second bandage includes a second light blocking layer, and the second bandage is coupled to the first bandage via adhering the second light blocking layer to the first light blocking layer.

Example 5: The sensor according to example 4, wherein the first bandage includes a tear prevention portion extending from the first light blocking layer.

Example 6: The sensor according to example 5, wherein the first light blocking layer and the tear prevention portion include a metallized tape.

Example 7: The sensor according to any of examples 1 to 6, wherein the sensor is asymmetrical about a midline that extends in a lateral direction of the sensor.

Example 8: The sensor according to any of examples 1 to 7, wherein the first bandage is configured to adhere to itself via the first type of adhesive and the second bandage is configured to adhere to a patient to facilitate application of the sensor to the patient.

Example 9: The sensor according to any of examples 1 to 8, including a flexible circuit with the LED and the detector, wherein at least a portion of the flexible circuit with the LED and the detector is positioned between the first bandage and the second bandage.

Example 10: The sensor according to example 9, wherein the first bandage includes a first light blocking layer and the second bandage includes a second light blocking layer, and wherein the at least the portion of the flexible circuit with the LED and the detector is positioned between the first light blocking layer and the second light blocking layer.

Example 11: The sensor according to any of examples 1 to 10, wherein the second type of adhesive includes a silicone adhesive.

Example 12: The sensor according to any of examples 1 to 11, wherein the first bandage includes a marker disposed at an edge of the first bandage to facilitate recognition of the edge.

Example 13: A sensor, including: a light-emitting diode (LED); a detector to detect light emitted by the LED; and a body that supports the LED and the detector, wherein the body includes: a first bandage including a first zone of a first adhesive; and a second bandage including a second zone of a second adhesive.

Example 14: The sensor of claim 13, wherein the first bandage includes a backing formed from a porous material and the second bandage includes a metallized tape.

Example 15: The sensor of any of claim 13 or 14, wherein the second adhesive includes a silicone adhesive.

Example: 16: The sensor of any of claims 13 to 15, wherein the first adhesive includes an acrylic adhesive.

Example 17: The sensor of any of claims 13 to 16, wherein the first bandage includes a tear prevention portion that includes a metallized tape around at least a portion of an outer perimeter of the first bandage.

Example 18: The sensor of any of claims 13 to 17, wherein the sensor is asymmetrical about a midline that extends in a lateral direction of the sensor.

Example 19: A system, including: a sensor including a detector to generate sensor data indicative of a physiological parameter of a patient, wherein the sensor includes a body to support the detector, and wherein the body includes: a first bandage including a first zone of a first adhesive; and a second bandage including a second zone of a second adhesive; and a monitor communicatively coupled to the sensor to receive and process the sensor data to determine the physiological parameter of the patient.

Example 20: The system of claim 19, wherein the first adhesive includes an acrylic adhesive, and the second adhesive includes a silicone adhesive.

What is claimed is:

1. A sensor, comprising:
a light-emitting diode (LED);
a detector to detect light emitted by the LED; and
a body that supports the LED and the detector, wherein the body comprises:
a first bandage comprising a respective surface with a first type of adhesive; and
a second bandage coupled to a portion of the first bandage and comprising a respective surface with a second type of adhesive.

2. The sensor of claim 1, wherein the first bandage comprises a backing, and wherein the backing comprises:
the respective surface with the first type of adhesive; and
a porous material with through holes that extend through the first type of adhesive.

3. The sensor of claim 2, wherein the porous material comprises polyethylene, polypropylene, or any combination thereof.

4. The sensor of claim 2, wherein the first bandage comprises a first light blocking layer coupled to the backing, the second bandage comprises a second light blocking layer, and the second bandage is coupled to the first bandage via adhering the second light blocking layer to the first light blocking layer.

5. The sensor of claim 4, wherein the first bandage comprises a tear prevention portion extending from the first light blocking layer.

6. The sensor of claim 5, wherein the first light blocking layer and the tear prevention portion comprise a metallized tape.

7. The sensor of claim 1, wherein the sensor is asymmetrical about a midline that extends in a lateral direction of the sensor.

8. The sensor of claim 1, wherein the first bandage is configured to adhere to itself via the first type of adhesive and the second bandage is configured to adhere to a patient to facilitate application of the sensor to the patient.

9. The sensor of claim 1, comprising a flexible circuit with the LED and the detector, wherein at least a portion of the flexible circuit with the LED and the detector is positioned between the first bandage and the second bandage.

10. The sensor of claim 9, wherein the first bandage comprises a first light blocking layer and the second bandage comprises a second light blocking layer, and wherein the at least the portion of the flexible circuit with the LED and the detector is positioned between the first light blocking layer and the second light blocking layer.

11. The sensor of claim 1, wherein the second type of adhesive comprises a silicone adhesive.

12. The sensor of claim 1, wherein the first bandage comprises a marker disposed at an edge of the first bandage to facilitate recognition of the edge.

13. A sensor, comprising:
a light-emitting diode (LED);
a detector to detect light emitted by the LED; and
a body that supports the LED and the detector, wherein the body comprises:
a first bandage comprising a first zone of a first adhesive; and
a second bandage comprising a second zone of a second adhesive.

14. The sensor of claim 13, wherein the first bandage comprises a backing formed from a porous material and the second bandage comprises a metallized tape.

15. The sensor of claim 13, wherein the second adhesive comprises a silicone adhesive.

16. The sensor of claim 15, wherein the first adhesive comprises an acrylic adhesive.

17. The sensor of claim 13, wherein the first bandage comprises a tear prevention portion that comprises a metallized tape around at least a portion of an outer perimeter of the first bandage.

18. The sensor of claim 13, wherein the sensor is asymmetrical about a midline that extends in a lateral direction of the sensor.

19. A system, comprising:
a sensor comprising a detector to generate sensor data indicative of a physiological parameter of a patient, wherein the sensor comprises a body to support the detector, and wherein the body comprises:
a first bandage comprising a first zone of a first adhesive; and
a second bandage comprising a second zone of a second adhesive; and
a monitor communicatively coupled to the sensor to receive and process the sensor data to determine the physiological parameter of the patient.

20. The system of claim 19, wherein the first adhesive comprises an acrylic adhesive, and the second adhesive comprises a silicone adhesive.
